(19) United States
(12) Patent Application Publication
(10) Pub. No.: US 2017/0367561 A1
(43) Pub. Date: Dec. 28, 2017

(54) CAPSULE ENDOSCOPE, IMAGE PROCESSING SYSTEM INCLUDING THE SAME AND IMAGE CODING DEVICE INCLUDED THEREIN

(71) Applicant: Electronics and Telecommunications Research Institute, Daejeon (KR)

(72) Inventor: Seong Mo PARK, Daejeon (KR)

(21) Appl. No.: 15/458,881

(22) Filed: Mar. 14, 2017

(30) Foreign Application Priority Data
Jun. 24, 2016 (KR) 10-2016-0079578

(51) Int. Cl. A61B 1/00; A61B 1/04; H04B 13/00; G02B 23/24; A61B 1/06; H04N 5/225

(52) U.S. Cl. CPC A61B 1/00016; A61B 1/06; H04N 5/2256; H04B 13/005; G02B 23/2484; A61B 1/00009; A61B 1/041; A61B 1/00045; A61B 1/00025; H04N 2005/2255

(57) ABSTRACT

Provided is a capsule endoscope that includes a light source configured to emit light to an internal surface of an organ of a human body, an image sensor configured to use light reflected from the internal surface of the organ to generate image data, a processor configured to perform coding on the image data to generate coded data, and perform logical operation on the coded data and a binary code to generate final data, and a communication circuit configured to output the final data to an outside of the human body.

### CROSS-REFERENCE TO RELATED APPLICATIONS

The patent application claims priority under 35 U.S.C. §119 of Korean Patent Application No. 10-2016-0079578, filed on Jun. 24, 2016, the entire contents of which are hereby incorporated by reference.

### TECHNICAL FILED

The present disclosure herein relates to a capsule endoscope, and more particularly, to a capsule endoscope that uses an image coding device to process a captured image.

### DESCRIPTION OF THE RELATED ART

A capsule endoscope refers to a pill-shaped micro endoscope that has a diameter of about 9 mm to about 11 mm and a length of about 24 mm to about 26 mm. When a patient swallows the capsule endoscope, the capsule endoscope may image the inside of organs such as stomach, small intestine and large intestine. Doctors may directly examine the inside of the organs through a video screen or computer monitor. Since the capsule endoscope is significantly small, it is possible to relieve feeling of irritation and pain that patients have felt during typical endoscope examination.

The capsule endoscope employs a wireless communication technique in order to transmit captured image data. In order to use the wireless communication technique, the capsule endoscope transmits image data by using a high-frequency signal. However, since in order to transmit the image data by using the high-frequency signal, the capsule endoscope needs to have a modulation circuit, its volume may increase.

For the above reasons, the capsule endoscope employs a human-body communication technique in order to transmit the captured image data. The human-body communication may generate a current inside a human body to transmit image data to the outside of the human body. Since the human-body communication transmits data by using the human body as a medium, it does not need the high-frequency signal. However, the data rate of the human-body communication is slower than that of the wireless communication.

### SUMMARY

The present disclosure provides a capsule-type device that performs coding and logical operation on captured image data to generate final data and output the generated final data to the outside of a human body, an image processing system including the same, and an image coding device included therein.

A capsule endoscope, image processing system including the same and image coding device included therein according to embodiments of the inventive concept include a light source, image sensor, processor and communication circuit.

The light source emits light to an internal surface of an organ of a human body, the image sensor receives light reflected from the internal surface of the organ to generate image data, the processor generates coded data by coding the image data provided from the image sensor and generates final data by performing logical operation on the coded data and a binary code, and the communication circuit outputs the final data to an outside of the human body.

An image processing system according to an embodiment of the inventive concept includes a capsule endoscope and reception device.

The capsule endoscope generates first image data based on an image inside an organ, performs coding on the first image data to generate coded data, and performs a logical operation on the coded data and a binary code to generate final data, and the reception device generates recovery data by performing the logical operation on the final data provided from the capsule endoscope and the binary code and to generate second image data by decoding corresponding to the coding on the recovery data.

A capsule endoscope, image processing system including the same and image coding device included therein according to embodiments of the inventive concept include an image sensor, image processor and communication circuit.

The image sensor receives light from an outside to generate image data, the image processor generates coded data by coding the image data provided from the image sensor and generates final data by performing a logical operation on the coded data and a binary code, and the a communication circuit outputs the final data.

### BRIEF DESCRIPTION OF THE FIGURES

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a block diagram that shows an image processing system including a capsule endoscope according to an embodiment of the inventive concept;

FIG. 2 is a block diagram that shows the capsule endoscope in FIG. 1;

FIG. 3 is a block diagram that shows an image processor in FIG. 2;

FIG. 4 is a conceptual view that shows a logical operation on data performed at a first logical operator in FIG. 3;

FIG. 5 is a block diagram that shows a reception device in FIG. 1;

FIG. 6 is a block diagram that shows a data recovery circuit in FIG. 5;

FIG. 7 is a flow chart that shows an image coding method of the capsule endoscope in FIG. 2;

FIG. 8 is a flow chart that shows an image decoding method of the reception device in FIG. 5; and

FIG. 9 is a conceptual view that shows a capsule endoscope according to an embodiment of the inventive concept.

### DETAILED DESCRIPTION

In the following, embodiments of the inventive concept are described clearly and in detail so that a person skilled in the art to which the inventive concept pertains may easily practice the inventive concept.

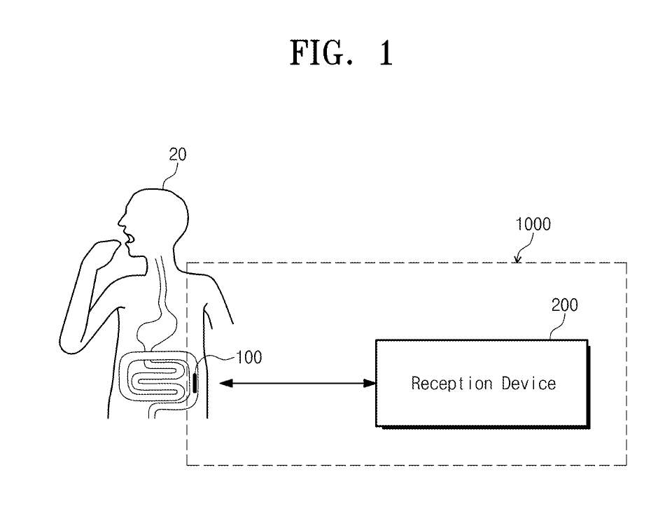

FIG. 1 is a block diagram that shows an image processing system including a capsule endoscope according to an embodiment of the inventive concept. An image processing system 1000 according to an embodiment of the inventive concept may include a capsule endoscope 100 and a reception device 200.

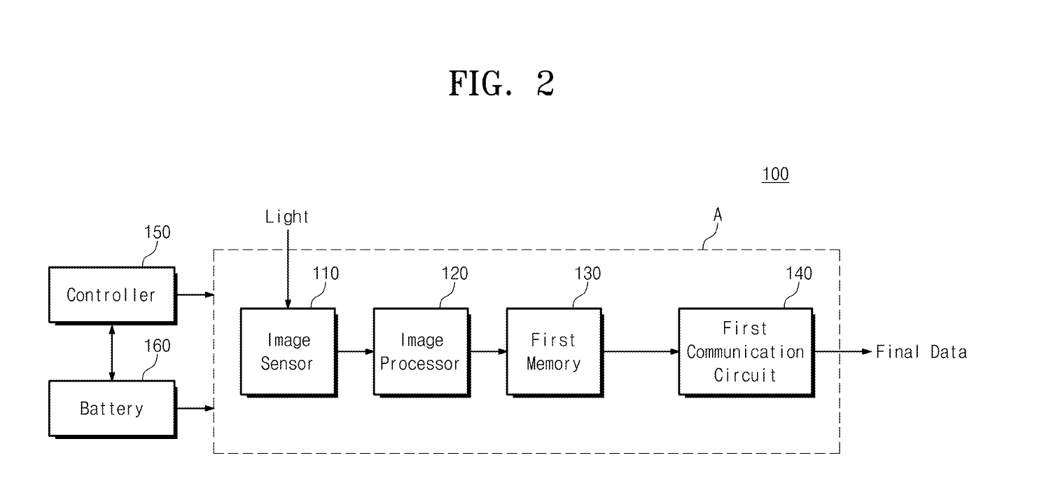

As shown in FIG. 2, a human being 20 may swallow the capsule endoscope 100 for endoscopy. When the human being 20 swallows the capsule endoscope 100, the capsule endoscope 100 may move the inside of an organ to image the internal surface of the organ to generate image data. As an example, the capsule endoscope 100 may start imaging from when entering the inside of the organ. As an example, the capsule endoscope 100 may start imaging from when entering the inside of the organ.

The capsule endoscope 100 may generate data by coding the captured image data. The capsule endoscope 100 may output the generated data to the outside of the human being 20. As an example, the capsule endoscope 100 may transmit the generated data to the reception device 200 that is installed outside the human being 20. The configuration of the capsule endoscope 100 is described with reference to FIGS. 2 to 4 below.

The reception device 200 may receive data from the capsule endoscope 100. The reception device 200 may decode the received data to generate image data. As an example, the reception device 200 may be implemented in at least one of a personal computer, desktop, laptop, tablet computer, digital camera, camcorder, smart phone, mobile device, and wearable device.

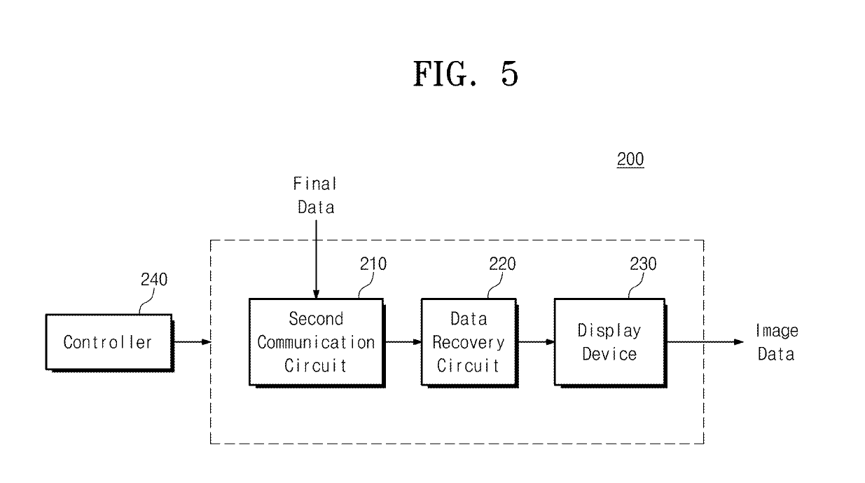

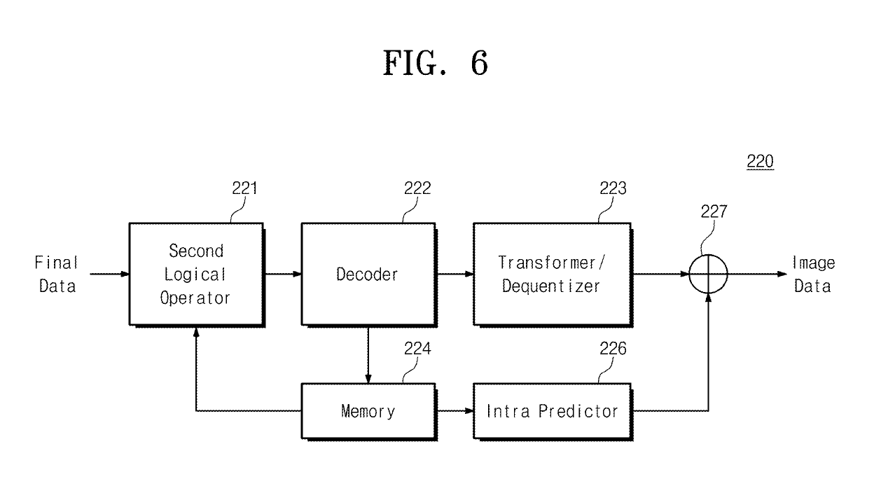

The reception device 200 may display image data. In addition, the reception device 200 may analyze and read the image data. As an example, the reception device 200 may implement various functions, such as image enlargement, continuous-viewing, and edition by using an image reader. The configuration of the reception device 200 is described in detail with reference to FIGS. 5 and 6.

FIG. 2 is a block diagram that shows the capsule endoscope in FIG. 1. Referring to FIGS. 1 and 2, the capsule endoscope 100 may include an image coding device A, a controller 150, and a battery 160.

The image coding device A may include an image sensor 110, an image processor 120, a first memory 130, and a first communication circuit 140. The image coding device A may be included in the capsule endoscope 100 for image data processing. The embodiment is not limited thereto and the image coding device A may be included in at least one of the personal computer, desktop, laptop, tablet computer, digital camera, camcorder, smart phone, mobile device, and wearable device, for the image data processing.

The image sensor 110 may receive light from a lens (not shown). The image sensor 110 may be one of a charge coupled device (CCD) and complementary metal-oxide semiconductor (CMOS). As an example, it is assumed that the image sensor 110 is the CCD. The image sensor 110 incorporates a plurality of photo-diode elements. When light enters the plurality of photo-diodes, each of the plurality of photo-diodes may generate an electron according to an amount of incident light. The image sensor 110 may generate image data based on the amount of electron generated.

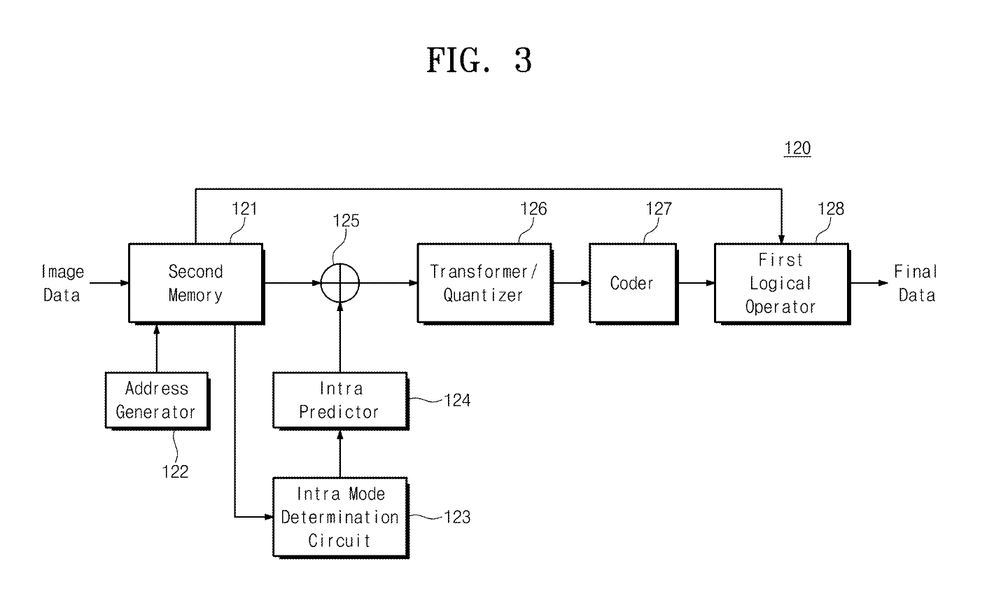

The image processor 120 may receive the image data from the image sensor 110. The image processor 120 may use the image data to generate residual data, and process the generated residual data. The image processor 120 may convert and quantize the residual data. The conversion and quantization of the residual data and image data are further described with reference to FIG. 3.

The image processor 120 may perform coding and a logical operation by using the converted and quantized residual data. The image processor 120 may transmit, to the first memory 130, final data that is generated through the coding and the logical operation process. The embodiment is not limited thereto and the image processor 120 may transmit the final data directly to the first communication circuit 140. The configuration of the image processor 120 is described with reference to FIG. 3.

The first memory 130 may receive the final data from the image processor 120. The memory 130 may store the final data. The first memory 130 may be at least one of a nonvolatile memory and volatile memory. In the case where the first memory 130 is the nonvolatile memory, the memory may store data that needs preservation. As an example, the first memory 130 may include a NAND-type flash memory, phase-change RAM (PRAM), magneto-resistive RAM (MRAM), resistive RAM (ReRAM), ferro-electric RAM (FRAM), and NOR-type flash memory.

Alternatively, the first memory 130 may include a memory of a different kind together. As an example, the first memory 130 may include at least one of a static random access memory (SRAM), dynamic RAM (DRAM), and synchronous dynamic random access memory (SDRAM) that may temporarily store data, in addition to the nonvolatile memory. The first memory 130 may output stored final data in response to the control of the controller 150. The embodiment is not limited thereto and the first memory 130 may regularly output the stored final data. Alternatively, the first memory 130 may transmit final data to the first communication circuit in response to an input external request.

The first communication circuit 140 may output received final data to the outside of a human body. The first communication circuit 140 may receive a data request from the reception device 200, and provide final data to the reception device 200 in response thereto. Alternatively, the first communication circuit 140 may provide the received final data to the reception device 200 in real time.

The controller 150 may control the general operations of the image processor 120, the first memory 130, the first communication circuit 140, and the battery 160. In addition, the battery 160 may supply power for the actuation of the capsule endoscope 100. In order for the controller 150 to perform a control operation, the battery 160 may continuously supply power to the controller 150. As an example, the battery 160 may supply power in response to the control of the controller 150. When the capsule endoscope 100 arrives at a desired location for performing imaging, the controller 150 may control the power supply of the battery 160 in order to supply power to the image sensor 110, the image processor 120, the first memory 130, and the first communication circuit 140.

As another example, by the control of the controller 150, the battery 160 may supply power to the image sensor 110, the image processor 120, and the first memory 130 for a first time. In addition, after image is performed for the first time, the battery 160 may supply power to the first communication circuit 140 during a second time so that final data may be output to outside. As such, the controller 150 may control the power supply of the battery 160 in order to extent the imaging time of the capsule endoscope 100.

FIG. 3 is a block diagram that shows the image processor in FIG. 2. Referring to FIG. 3, the image processor 120 may include a second memory 121, an address generator 122, an intra mode determination circuit 123, an intra predictor 124, an adder 125, a transformer/quantizer 126, a coder 127, and a first logical operator 128.

The second memory 121 may receive image data from the image sensor 110. The second memory 121 may be a nonvolatile memory. As an example, the second memory 121 may include at least one of the NAND flash memory, PRAM, MRAM, ReRAM, FRAM and NOR flash memory. The second memory 121 may receive an address from the address generator 122. The second memory 121 may selectively output final data according to the received address. Also, the second memory 121 may store a binary code needed for logical operation.

The address generator 122 may generate an address according to the control of the controller 150. The address generator 122 may provide the generated address to the second memory 121.

The intra mode determination circuit 123 may determine a mode for performing intra prediction. As an example, the intra mode determination circuit 123 may select at least one of nine prediction modes in order to decrease the difference between a prediction block and a block to be coded. The intra mode determination circuit 123 may transmit, to the intra predictor 124, information on the selected prediction mode among the nine prediction modes.

The intra predictor 124 may perform intra prediction on image data on a macro block basis. The macro block is a process unit of an image compression format. As an example, the macro block may have a size of 4×4 or 16×16. The intra predictor 124 may use a macro block adjacent to the prediction target macro block of the current frame of the image data to obtain prediction data on the prediction target macro block. That is, intra prediction may be performed based on macro blocks that are included in a single frame. The intra predictor 124 may generate prediction data by the intra prediction.

The adder 125 may receive image data from the second memory 121, and receive prediction data from the intra predictor 124. The adder 125 may add the image data and the prediction data. The adder 125 may generate residual data based on the prediction data and the image data. The residual data may be generated as a result of the operation between the prediction data and the image data. The adder 125 may provide the residual data to the transformer/quantizer 126.

The transformer/quantizer 126 may receive the residual data. The transformer/quantizer 126 may transform the residual data to frequency-domain data and quantize the frequency-domain data. As an example, the transformation may be one of discrete cosine transform (DCT), discrete sine transform (DST), and integer transform. The transformer/quantizer 126 may provide the transformed and quantized residual data to the coder.

The coder 127 may receive the transformed and quantized residual data. The coder 127 may perform coding on the transformed and quantized residual data. As an example, the coding may be entropy coding. As an example, the entropy coding may be one of Huffman coding, arithmetic coding, range encoding, universal coding, Shannon-Fano coding, and Tunstall coding. The coder 127 may generate coded data using the entropy coding. The coder 127 may provide the generated coded data to the first logical operator 128.

The first logical operator 128 may receive the coded data. The first logical operator 128 may perform a logical operation on the coded data. As an example, the first logical operator 128 may perform the logical operation on a binary code and the coded data. The first logical operator 128 may receive the binary code from the second memory 121. The logical operation method of the first logical operator 128 is described with reference to FIG. 5. The first logical operator 128 may use the logical operation to generate final data. The first logical operator 128 may provide the generated final data to the first memory 130.

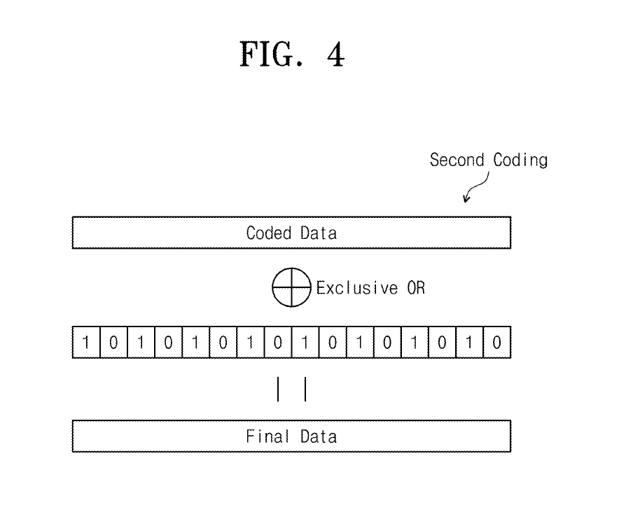

FIG. 4 is a conceptual view that shows a logical operation on data performed at the first logical operator in FIG. 3. Referring to FIGS. 3 and 4, the first logical operator 128 may perform exclusive OR (XOR) operation on the coded data and any binary code. As an example, any binary code may be a 16-bit code in which digits “1” and “0” are alternately arranged. The embodiment is not limited thereto, and any binary code may be configured in various forms.

Since human-body communication is a communication method in which a human body is used as a medium, there is the probability that an error occurs. Also, the coded data in which the same values are continuously arranged may be vulnerable to an error that may occur in a communication process. By performing XOR operation on the coded data and the binary code, generated final data may experience a decrease in the continuous arrangement of the same values. Thus, the final data has high resistance to an error. In addition, since the XOR operation needs no complicated operation, the first logical operator 128 consumes low power.

The capsule endoscope 100 according to an embodiment of the inventive concept may generate final data by performing entropy coding and XOR operation on image data. Since the capsule endoscope 100 transmits the image data in the form of final data, the probability that an error occurs may decrease.

FIG. 5 is a block diagram that shows the reception device in FIG. 1. The reception device 200 may include a second communication circuit 210, a data recovery circuit 220, a display unit 230, and a controller 240.

The second communication circuit 210 may communicate with the capsule endoscope 100. As an example, the second communication circuit 210 may receive final data from the capsule endoscope 100. Also, the second communication circuit 210 may request the final data from the capsule endoscope 100 in response to the control signal of the controller 240. The second communication circuit 210 may deliver the received final data to the data recovery circuit 220.

The data recovery circuit 220 may decode the final data. The data recovery circuit 220 may perform a logical operation and decoding on the final data to generate decoded data. In addition, the data recovery circuit 220 may recover residual data by performing inverse transformation and dequantization on the decoded data. In addition, the data recovery circuit 220 may perform intra prediction on the decoded data. The data recovery circuit 220 may use the residual data and the intra-predicted data to output image data to the display 230. The structure of the data recovery circuit 220 is described with reference to FIG. 6.

The display 230 may display the image data. As an example, the display 230 may be implemented in one of a liquid crystal display (LCD), organic light emitting diode (OLED) display, active matrix OLED (AMOLED) display, and LED.

The controller 240 may control the operations of the second communication circuit 210, the data recovery circuit 220, and the display unit 230. As an example, the controller 240 may generate a control signal for requesting final data from the capsule endoscope 100. The generated control signal may be provided to the capsule endoscope 100 through the second communication circuit 210.

FIG. 6 is a block diagram that shows the data recovery circuit in FIG. 5. The data recovery circuit t 220 may include a second logical operator 221, a decoder 222, an inverse transformer/dequantizer 223, a memory 224, an intra predictor 226, and an adder 227.

The second logical operator 221 may use a binary code used for the logical operation in the capsule endoscope 100 to perform the logical operation. At this point, the second logical operator 221 may receive the binary code from the memory 224. As an example, the second logical operator 221 may perform XOR operation on final data and the binary code. The second logical operator 221 may perform XOR operation to generate recovery data. In the case where there is no communication error, the recovery data may be the same as the coded data of the capsule endoscope 100. The second logical operator 221 may provide the recovery data to the decoder 222.

The decoder 222 may generate decoded data by decoding the recovery data. The decoder 222 may provide the decoded data to the inverse transformer/dequantizer 223.

The inverse transformer/dequantizer 223 may inversely transform the decoded data and dequantize the inversely transformed data. Accordingly, the inverse transformer/dequantizer 223 may recover the residual data. The inverse transformer/dequantizer 223 may provide the residual data to the adder 227.

The memory 224 may store the decoded data. As an example, the memory 224 may be a nonvolatile memory. The memory 224 may provide the decoded data to the intra predictor 226. In FIG. 6, the memory 224 is installed outside the decoder 222 and the intra predictor 226. However, the memory 224 may be included in one of the decoder 222 and the intra predictor 226.

The intra predictor 226 may generate prediction data by performing intra prediction on the decoded data. The configuration and function of the intra predictor 226 are those of the intra predictor 124 in FIG. 4.

The adder 227 may receive residual data recovered by the inverse transformer/dequantizer 223 and prediction data generated by the intra predictor 226. The adder 227 may add the residual data and the prediction data. The adder 227 may provide data corresponding to a result of operation (e.g., image data) to the display 230.

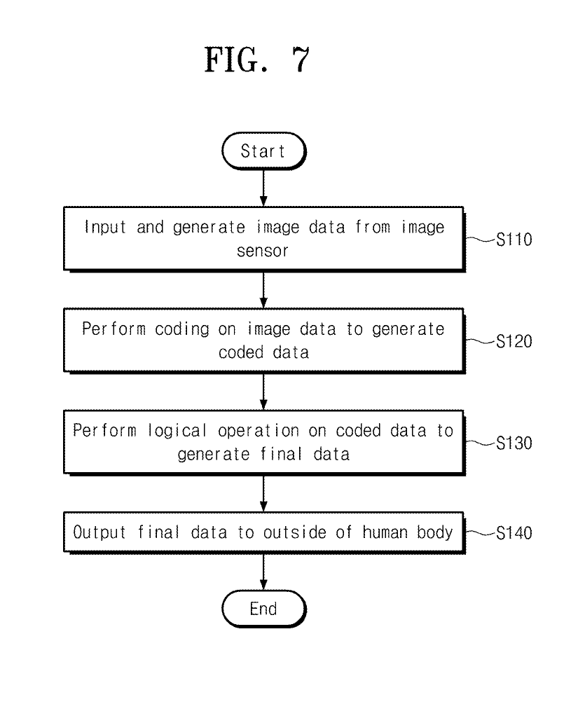

FIG. 7 is a flow chart that shows an image coding method of the capsule endoscope in FIG. 2. Referring to FIGS. 2 and 7, the image sensor 110 of the capsule endoscope 100 receives light through a lens in step S110. The image sensor 110 may use the received light to generate image data.

In step S120, the capsule endoscope 100 may perform coding on the image data. The coding may be performed by the image processor 120 of the capsule endoscope 100. As an example, the coding may be entropy coding. The image processor 120 may generate coded data by coding the image data.

In step S130, the capsule endoscope 100 may perform a logical operation on the coded data. The logical operation may be performed by the image processor 120 of the capsule endoscope 100. As an example, the logical operation may be XOR operation. The image processor 120 may generate final data by performing the logical operation on the coded data.

In step S140, the capsule endoscope 100 may output final data to the outside of a human body. The final data may be output through the first communication circuit 140 of the capsule endoscope 100. The first communication circuit 140 may output the final data by using a human-body communication method.

Referring to FIG. 7, the capsule endoscope 100 according to an embodiment of the inventive concept may generate final data by performing coding and logical operation on image data. Accordingly, the final data may have high resistance to an error that may occur in human-body communication.

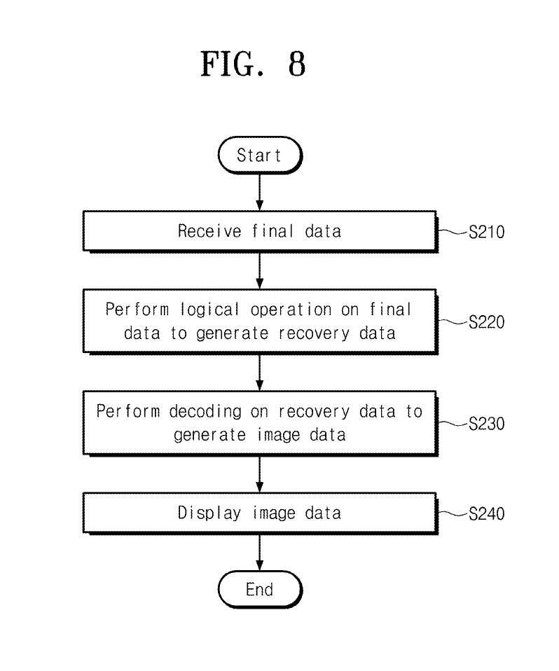

FIG. 8 is a flow chart that shows an image decoding method of the reception device in FIG. 5. Referring to FIGS. 5 and 8, in step S210, the reception device 200 may receive final data. As an example, the reception device 200 may receive the final data through the second communication circuit 210.

In step S220, the reception device 200 may perform a logical operation on the final data. The logical operation may be performed by the data recovery circuit 220 of the reception device 200. As an example, the logical operation may be XOR operation. The data recovery circuit 220 may generate recovery data by performing the logical operation on the final data.

In step S230, the reception device 200 may perform decoding on the recovery data. The decoding may be performed by the data recovery circuit 220 of the reception device 200. As an example, the decoding may be entropy decoding. The data recovery circuit 220 may perform decoding on the recovery data to generate decoded data. In addition, the data recovery circuit 220 may generate image data using the decoded data.

In step S240, the reception device 200 may display the image data. The display 230 of the reception device 200 may display the image data.

The reception device 200 according to an embodiment of the inventive concept may perform the logical operation and decoding on the final data that is received from the capsule endoscope 100. Accordingly, it is possible to provide recovered image data through the display 230.

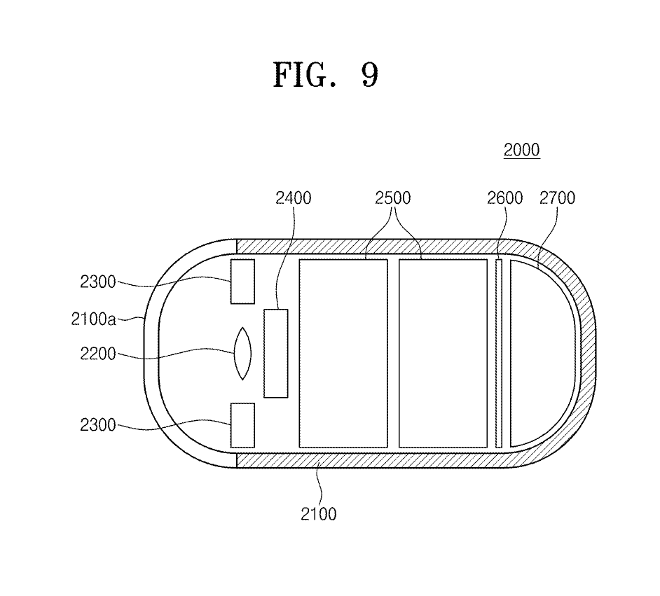

FIG. 9 is a conceptual view that shows a capsule endoscope according to an embodiment of the inventive concept. A capsule endoscope 2000 may include capsule portions 2100 and 2100a, a lens 2200, a light source 2300, an image sensor 2400, a power source 2500, a processor 2600, and a communication circuit 2700.

The capsule portions 2100 and 2100a may be formed from a material harmless to a human body. As an example, the capsule portion 2100a of the capsule portions 2100 and 2100a that surrounds the lens 2200 may be formed from a semi-spherical transparent material that is in the shape of an optical dome. As an example, the capsule portion 2100a may be a transparent plastic material. The capsule portions 2100 and 2100a may include the lens 2200, the light source 2300, the image sensor 2400, the power source 2500, the processor 2600, and the communication circuit 2700 therein. As an example, there may be a space between the capsule portion 2100a and the lens 2200. Thus, even when an organ contracts, the lens 2200 may perform imaging while maintaining a certain distance from the inner wall of the organ.

The lens 2200 may receive light reflected from the internal surface of an organ inside a human body. As an example, the lens 2200 for endoscope may be a short focal length lens that includes a small aperture. The light source 2300 may be located around the lens 2200. As an example, the light source 2300 may be an LED. There may be included one or more light source 2300. Since the inside of an organ is dark, there may be a need for the light source 2300 for endoscopy. While the light source 2300 emits light, it may illuminate the inside of the organ. As an example, the light source 2300 may regularly emit light.

The image sensor 2400 may obtain light received from the lens 2200. The image sensor 2400 is similar or the same as the image sensor 110 in FIG. 2. The image sensor 2400 may generate image data. The image sensor 2400 may deliver the generated image data to the processor 2600.

The power source 2500 may supply power for the actuation of the capsule endoscope 2000. The power source 2500 may supply power to the light source 2300, the image sensor 2400, the processor 2600, and the communication circuit 2700.

The processor 2600 may perform various logical operations and/or logical operation in order to process operations. To this end, the processor 2600 may include one or more processor cores. As an example, the processor core of the processor 2600 may include a special purpose logic circuit (e.g., field programmable gate array (FPGA), an application specific integrated chip (ASIC) or the like).

The processor 2600 may be similar or the same as the image processor 120 in FIG. 2. The processor 2600 may receive image data from the image sensor 2400. The processor 2600 may process the received image data. Since the operation of the processor 2600 has been described with reference to FIG. 2, a detailed description is omitted. The processor 2600 may deliver, to the communication circuit 2700, final data that is generated through the processing of the image data.

The communication circuit 2700 may receive the final data from the processor 2600. The communication circuit 2700 may transmit the final data to the outside of a human body through human-body communication.

According to an embodiment of the inventive concept, data loss may decrease and image processing efficiency may be enhanced when image data inside an organ is transmitted to the outside of a human body.

The above-described details are particular examples for practicing the inventive concept. The inventive concept would include not only the above-described embodiments but also embodiments that may be simply changed in design or easily changed. Also, the inventive concept would also include techniques that may be practiced through an easy variation in the future by the using of the above-described embodiments.

What is claimed is:

1. A capsule endoscope comprising:
a light source configured to emit light to an internal surface of an organ of a human body;
an image sensor configured to receive light reflected from the internal surface of the organ to generate image data;
a processor configured to generate coded data by coding the image data, and generate final data by performing a logical operation on the coded data and a binary code; and
a communication circuit configured to output the final data to an outside of the human body.

2. The capsule endoscope of claim 1, further comprising a lens configured to receive the reflected light.

3. The capsule endoscope of claim 2, further comprising a capsule portion configured to cover the light source, the image sensor, the processor, and the communication circuit,
wherein a part of the capsule portion that surrounds the lens is formed from a transparent material.

4. The capsule endoscope of claim 1, further comprising a battery configured to supply power to at least one of the light source, the image sensor, the processor, or the communication circuit.

5. The capsule endoscope of claim 4, wherein the battery is configured to supply the power to the light source, the image sensor, the processor, and the communication circuit when the capsule endoscope arrives at a target location.

6. The capsule endoscope of claim 4, wherein the battery is configured to supply the power to the light source, the image sensor, and the processor during a first time interval, and supply the power to the communication circuit during a second time interval following the first time interval.

7. The capsule endoscope of claim 1, wherein the communication circuit is configured to operate with human-body communication using the human body as a medium to output the final data to the outside of the human body.

8. The capsule endoscope of claim 1, wherein the coding performed by the processor includes entropy coding.

9. The capsule endoscope of claim 1, wherein the logical operation performed by the processor includes an exclusive OR (XOR) operation, and
the processor is configured to generate the final data by performing the XOR operation on the coded data and the binary code.

10. An image processing system comprising:
A capsule endoscope configured to,
generate first image data based on an image inside an organ,
perform coding on the first image data to generate coded data, and
perform a logical operation on the coded data and a binary code to generate final data; and
a reception device configured to,
generate recovery data by performing the logical operation on the final data and the binary code, and
generate second image data by decoding corresponding to the coding the recovery data.

11. The image processing system of claim 10, wherein the reception device comprises:
a logical operator configured to generate the recovery data by performing the logical operation on the final data and the binary code;
a decoder configured to generate the second image data by performing the decoding the recovery data; and
a display device configured to display the second image data.

12. The image processing system of claim 10, wherein the logical operation includes an exclusive OR (XOR) operation.

13. An image coding device comprising:
an image sensor configured to receive light from an outside to generate image data;
an image processor configured to generate coded data by coding the image data, and generate final data by performing a logical operation on the coded data and a binary code; and
a communication circuit configured to output the final data.

14. The image coding device of claim 13, wherein the image processor comprises:
an intra predictor configured to generate prediction data by performing intra prediction on the image data;
an adder configured to generate residual data based on the image data and the prediction data;
a transformer/quantizer configured to transform and quantize the residual data to generate transformed and quantized residual data;
a coder configured to generate the coded data by coding the transformed and quantized residual data; and
a logical operator configured to generate the final data by performing the logical operation on the coded data and the binary code.

15. The image coding device of claim 14, wherein the coding performed by the coder includes entropy coding.

16. The image coding device of claim 14, wherein the logical operation performed by the logical operator includes an exclusive OR (XOR) operation.

17. The image coding device of claim 16, wherein the logical operator is configured to perform the XOR operation on the coded data and the binary code to generate the final data.

18. The image coding device of claim 13, further comprising a memory configured to store the image data and the binary code.
